SEQUENCE LISTING (2 sequences)

SEQ ID NO: 1 - DNA, 47 bp, synthetic construct
  source 1..47: mol_type = other DNA
gatgagatagtagattccaccactatttttttttttttaagtggtgg

SEQ ID NO: 2 - RNA, 47 nt, synthetic construct
  source 1..47: mol_type = other RNA
gatgagatagtagattccaccactatttttttttttttaagtggtgg

(19) United States
(12) Patent Application Publication
(10) Pub. No.: US 2024/0175076 A1
(43) Pub. Date: May 30, 2024

(54) Quantum Dot-Nucleotide Based Ratiometric Fluorescent Molecular Beacon for Quantitative CRISPR/Cas Activity Detection

(71) Applicant: The Government of the United States of America, as represented by the Secretary of the Navy, Arlington, VA (US)

(72) Inventors: Sebastián A. Díaz, Silver Spring, MD (US); Christopher M. Green, Lorton, VA (US); Joseph R. Spangler, La Plata, MD (US); Divita Mathur, College Park, MD (US); Kimihiro Susumu, Alexandria, VA (US); David A. Stenger, Annapolis, VA (US); Igor L. Medintz, Springfield, VA (US)

(21) Appl. No.: 18/346,973

(22) Filed: Jul. 5, 2023

Related U.S. Application Data
(60) Provisional application No. 63/358,719, filed on Jul. 6, 2022.

(51) Int. Cl. C12Q 1/6818; C12Q 1/6876; C12Q 1/6806

(52) U.S. Cl. CPC C12Q 1/6818; C12Q 1/6876; C12Q 1/6806

(57) ABSTRACT

A flexible strategy for assembling luminescent and colorimetric quantum dot/nucleic acid hairpin (QD-HP) molecular beacons for use in CRISPR/Cas diagnostics uses chimeric peptide/peptide nucleic acid (PNA) to conjugate fluorescently labeled DNA or RNA hairpins to ZnS-coated QDs.

### CROSS-REFERENCE TO RELATED APPLICATIONS

This application claims the benefit of U.S. Provisional Patent Application No. 63/358,719 filed Jul. 6, 2022, the entirety of which is incorporated herein by reference.

This application is related to the commonly-owned U.S. patent application Ser. No. 18/203,189 filed May 30, 2023.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; techtran@nrl.navy.mil, referencing NC 210911.

### INCORPORATION BY REFERENCE

This application incorporates by reference the Sequence Listing XML file submitted herewith via the patent office electronic filing system having the file name 210911-sequences.xml” and created on Jun. 29, 2023 with a file size of 2,899 bytes.

### BACKGROUND

The CRISPR/Cas system evolved in bacteria as a defense mechanism against viral infection; its discovery resulted in the 2020 Nobel prize. This system has become well-known due to the possibilities it presents for medical applications such as gene editing, with multiple companies now investigating its use in clinical applications. The CRISPR (clustered regularly interspersed short palindromic repeats) portion of the system is a pattern of nucleic acid sequences that are recognized and bound by Cas (CRISPR-associated protein) enzymes to activate the system. Varying levels of activity, target, and specificity exist within the family of CRISPR/Cas systems, with different Cas enzymes providing varying functionalities.

The well-known Cas9 binds to a guide RNA (gRNA) strand which contains a targeting domain that is complementary to a particular double-stranded (ds) DNA target. In the presence of the target dsDNA, the dsDNA is bound by the Cas9-gRNA complex and is subsequently cleaved. The ability to modify the targeting domain of the gRNA to bind and cleave any dsDNA sequence provides specificity that can be exploited for gene editing. The Cas12 and Cas13 systems work similarly, but with an additional activation that occurs upon gRNA binding to the complementary target strand, resulting in non-specific and rapid cleavage of single stranded DNA and RNA by Cas12 and Cas13, respectively. Though not of primary interest for gene-editing, the activation of the non-specific nucleotide cleavage in the presence of target nucleic acids allows Cas12 and Cas13 to be employed as extremely sensitive and specific sensors for nucleic acids. By tailoring the gRNA to be complementary to nucleotide oligomers of particular interest, i.e. disease biomarkers or bacterial or viral genomes, picomolar levels of detection are often achieved. Sensitivity can be further improved by the amplification of target RNA or DNA by recombinase polymerase amplification (RPA) prior to introduction of Cas12 or Cas13, with the possibility of the detection of attomolar levels of target. Accordingly, such an approach presents an attractive basis for diagnostic applications including the detection of nucleic acids indicating the presence of pathogens. At least two companies have received regulatory approval to use this technology to develop assays for viral detection. Significant interest exists in translating this technology to varying assay systems such as high throughput laboratory systems, small volume microfluidics, or economic lateral flow assays, each meeting particular needs and requiring its own optimization.

Detection of the resulting nucleotides typically involves the creation of an optical signal by cleavage of a molecular beacon (MB), which are traditionally fluorophore-quencher (FQ) pairs bound to opposite ends of a short DNA or RNA hairpin. The combination of strand amplification and molecular beacon readout provides accessible and sensitive qualitative assays, though sensitivity is achieved at the expense of quantitative capabilities. FQ-MBs have low luminosity, single color emission, and small Stokes shift between their excitation and emission maxima. Consequently, experiments using FQ molecular beacons require either sensitive optical equipment for readout or the use of large quantities of molecular beacon, which in turn necessitates strand amplification to enhance Cas activity. Many alternative readout strategies have been explored ranging from colorimetric assays, such as the coupling of Cas activity to an enzymatically induced pH change in the presence of a pH sensitive dye, to nanoparticle-based assays, such as the modulation of gold nanoparticle aggregation or the release of quantum dots (QDs) from magnetic beads, as well as electrochemical strategies monitoring the release of redox-active dyes from gold electrodes. While promising, these strategies introduce additional steps and equipment for assay preparation, and have not outperformed molecular beacons.

A need exists for new, quantitative approaches for the detection of CRISP/Cas activity.

### BRIEF SUMMARY

Described herein is a flexible strategy for assembling luminescent and colorimetric quantum dot/nucleic acid hairpin (QD-HP) molecular beacons for use in CRISPR/Cas diagnostics. This strategy uses chimeric peptide/peptide nucleic acid (PNA) to conjugate fluorescently labeled DNA or RNA hairpins to ZnS-coated QDs.

In a first embodiment, a molecular beacon comprises a quantum dot (QD), a peptide-peptide nucleic acid (PNA) bound to the QD comprising a PNA sequence and peptide comprising a polyhistidine sequence for binding to the QD, and a nucleic acid hairpin strand bound to the PNA sequence and comprising a nucleic acid having a sequence complementary to the PNA sequence, an exposed loop substrate for a Cas enzyme, and a quencher configured as a Forster resonance energy transfer (FRET) partner of the QD, wherein the combination of the peptide-PNA and the nucleic acid hairpin strand locates the quencher in a position to quench QD fluorescence.

In a second embodiment, a method of detecting Cas enzyme activity comprises providing a molecular beacon according to the first embodiment, contacting the molecular beacon with a Cas enzyme associated with a guide RNA which is in turn paired with a target RNA, thus activating the Cas enzyme; and detecting a change in fluorescence from the QD, wherein the change results from cleavage of the exposed loop substrate by the activated Cas enzyme, resulting in dissociation of the quencher from the molecular beacon. It is expected that this method could be used to detect practically any CRISPR/Cas proteins having DNase and RNAse collateral activity as well as second messenger Cas-associated proteins showing collateral cleavage (e.g. Csm6).

In further embodiments, the method of the second embodiment is performed in multiplex with at least two different exposed loop substrate sequences, each associated with a different form QD (e.g., QDs of different sizes).

### BRIEF DESCRIPTION OF THE DRAWINGS

FIGS. 1A-1F show the design and characterization of the QD-HP (quantum dot/nucleic acid hairpin) reporters. FIG. 1A is a rendering of a QD-DHP (quantum dot/DNA hairpin) reporter depicting QD525 and surface ligands (grey) bound to a peptide-PNA (red) and DHP-Cy3 (black/yellow) duplex. QD525 is a CdSe/CdS/ZnS core/shell/shell QD with a diameter of 4.1±0.5 nm and an emission peak near 528 nm. FIGS. 1B and 1C show schematic illustrations of exemplary reporters configured for Cas12a (left) and Cas13a (right). FIG. 1D provides a schematic depicting the initial state of the QD-HP reporter (left), and the reporter complex following cleavage by a target-bound Cas RNP nuclease (right). Prior to cleavage, the QD is quenched by the surrounding dyes through Forster resonance energy transfer (FRET), inducing FRET-sensitized emission from the dyes. Upon cleavage, the dyes are released from the complex, and the QD fluorescence is recovered. FIG. 1E is a plot of the emission spectra of QD525:DHP-Cy3 reporters ranging from 0-12 Cy3 per QD, achieved by varying the molar ratio of DHP-Cy3 to QD525 during preparation. The spectra were normalized by the isosbestic point at 554 nm. FIG. 1F provides plots of the FRET efficiency (E_{FRET}) vs the molar ratio of DHP-Cy3 to QD525 on the left axis (black) and the predicted FRET donor-acceptor distance on the right axis (red). FIG. 1G is a plot of the photoluminescence (PL) ratio of Cy3 at 604 nm to QD525 at 528 nm vs the molar ratio of DHP-Cy3 to QD525. From approximately 2-12 Cy3 per QD525, the PL ratio was linearly proportional to the molar ratio.

FIGS. 2A-2E illustrate the dependence of Lachnospiraceae bacterium Cas12a (LbCas12a) trans-cleavage on molar ratio and concentration of QD-DHP reporter. FIG. 2A schematically depicts the QD-DHP prepared at 2, 4, 6, 8, and 10 DHP-Cy3 per 1 QD525. FIGS. 2B to 2E show results from various reaction conditions. Activated LbCas12a were prepared by preassembling Cas12a stepwise with CRISPR RNA (crRNA) and target ssDNA in 1×NEBuffer 2.1r and 1 mM DTT. For varying DHP-Cy3 to QD525 molar ratios, the total QD concentration was held constant in one set of experiments (FIGS. 2B and 2C), and the DHP-Cy3 concentration was held constant in a second set of experiments (FIGS. 2D and 2E). The progress of each reaction was quantified by the PL ratio normalized to control samples lacking target ssDNA (FIGS. 2B and 2D). The fraction of DHP cleaved was approximated from the normalized PL ratios with the initial DHP concentration and assuming proportionality between the PL ratio and the acceptor to donor ratio.

FIGS. 3A-3F show Cas12a (FIGS. 3A to 3C) and Cas13a (FIGS. 3D to 3F) target dilutions and Limit of Detection. FIGS. 3A to 3C show target ssDNA dilutions for detection by activation of Cas12a and subsequent cleavage of QD525:DHP-Cy3 reporters with 6 to 1 Cy3 per QD. Progress curves of the average fluorescence of QD525 (FIG. 3A) and normalized PL ratio of Cy3 to QD525 normalized to the control (FIG. 3B) are shown over 2 h for target ssDNA concentrations from 0 to 5120 pM. FIG. 3C is a plot of the normalized PL ratios at 1 h vs target concentration. The limit of detection (approximately 40 pM) is indicated by the point at which the curve intercepts the 36 deviation from the control PL ratio. FIGS. 3D to 3F show target RNA dilutions for detection by activation of Cas13a and subsequent cleavage of QD525:RHP-Cy3 (RHP stands for RNA hairpin) reporters with 6 to 1 Cy3 per QD. Progress curves of the average fluorescence of QD525 FIG. 3C and normalized PL ratio of Cy3 to QD525 normalized to the control FIG. 3D are shown over 2 h for target RNA concentrations from 0 to 10000 pM. FIG. 3D is a plot of the normalized PL ratios at 1 h vs target concentration. The limit of detection (approximately 100 pM) is indicated by the point at which the curve intercepts the 36 deviation from the control PL ratio

FIGS. 4A-4F depict multiplexed detection of target RNA and DNA with distinct QD-HP reporters. FIG. 4A shows a rendering of the QD525:RHP-Cy3 and QD525:DHP-Cy5 complexes to scale. FIG. 4B provides a graphic depicting the individual components combined for the multiplexed assays. LbCas12a and LwCas13a (Cas13a from Leptotrichia wadei) were pre-assembled with their respective crRNAs, QD525 was pre-assembled with RHP-Cy3 at 6:1 Cy3:QD525, and QD625 was pre-assembled with DHP-Cy5 at 10:1 Cy5:QD625. All components were combined in a single mixture for the multiplexed assays. Four sample mixtures were prepared: FIG. 4C no target RNA or DNA, FIG. 4D 10 nM target RNA and no target DNA, FIG. 4E no target RNA and 10 nM target DNA, and FIG. 4F 10 nM target RNA and DNA. Activation of each reporter was indicated by a significant deviation of the PL ratio from 1 for a given reporter/target combination.

FIGS. 5A-5D show a cell phone camera assay with QD525-DHP and Cas12a. FIG. 5A provides an image of the detection setup in real time, consisting of a 365 nm UV lamp, IPHONE 13 Pro, and a simple cardboard box with holes for the lamp and phone camera. FIG. 5B depicts an image from the IPHONE of the UV illuminated plate at t=0 min. The magnified images on the right depict separation of the red and green channels from the color image, as well as generation of the R/G image by dividing the integer values of the red channel by those in the green channel. The arrows indicate the sample with target DNA and fully assembled QD525:DHP-Cy3 reporter. FIG. 5C shows an image from the IPHONE at t=60 min. The same magnification and channel extractions as those in FIG. 5B are shown, and all samples are identical. The arrow indicates the positive sample, and the difference in this sample can be observed in all extracted channels between times 0 and 60 min. FIG. 5D provides plots of the mean R/G signal from wells 5-8. Control samples indicate completely disassembled QD and DHP, and the QD-DHP+Target sample is the only sample containing both target DNA and the fully assembled QD-DHP reporter. The QD-DHP+Target sample demonstrated a large change in the R/G ratio over the course of imaging.

FIGS. 6A-6C depict additional variations of the molecular beacon. In FIG. 6A the use of a chimeric DNA/RNA hairpin (cRHP) is included which allows for functionality of both the Cas12 and Cas13 systems as readouts. Additionally it minimizes downstream non-specific cleavage by Cas13, this results in faster kinetics and lower limits of detection. The QDs in FIG. 6A are coated with blocking peptides to assure enzyme activity. In FIG. 6B the same system is shown without the presence of the blocking peptides but with a modified peptide-PNA with an extended peptide sequence, which includes extended histag sequences. This results in the elimination of the need for the blocking peptides seen in FIG. 6A. In FIG. 6C the system is studied in the presence of PEGylated ligands on the QD surface. This demonstrates that varying the surface ligands on the QD surface does not lead to inactivation of the molecular beacon.

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

### Overview

Described herein is a system useful in CRISPR/Cas diagnostics that involves using new molecular beacons capable of the quantitative detection of nucleic acids. The molecular beacons comprise luminescent and colorimetric quantum dot/nucleic acid hairpin (QD-HP) which in turn includes a chimeric peptide/peptide nucleic acid (PNA) to conjugate DNA or RNA hairpins to ZnS-coated QDs, making them capable of reporting on either Cas12 (DNA) or Cas13 (RNA) systems. The hairpins place a Forster resonance energy transfer (FRET) acceptor on the end of the oligonucleotide hairpin close to the QD surface resulting in efficient FRET, with each hairpin presenting a single stranded oligonucleotide loop that is cleaved by the activated Cas enzyme. When the Cas enzyme encounters a target nucleic acid that is complementary to its guide RNA, the enzyme becomes activated for nonspecific or “collateral” cleavage of nearby non-targeted nucleic acid—in this case the enzyme acts on the hairpin loop of the molecular beacon. Upon cleavage of the loop, the stem of the hairpin denatures, allowing the acceptor-oligo fragment to diffuse away from the QD surface, resulting in an increase of QD fluorescence and decrease of the acceptor fluorescence.

QDs are particularly promising alternatives for molecular beacons due to greater brightness, strong UV absorbance paired with large and tunable Stokes shifts, exceptional photostability, and the potential for multiplexing due to their sharp emission peaks. Using FRET, these ratiometric reporters detect targets at pM levels (without nucleotide amplification) for both target DNA and RNA, and furthermore can be multiplexed and detected using the cameras in common consumer phones. The flexibility of this system is imparted by the dual functionality of the QD as both a FRET donor and as a central nanoscaffold for arranging nucleic acids and fluorescent acceptors on its surface. This method also provides a generalized approach that could be applied for use in other CRISPR/Cas nuclease systems.

Peptide nucleic acids (PNAs) are synthetic mimics of DNA in which the deoxyribose phosphate backbone is replaced by a pseudo-peptide polymer to which the nucleobases are linked. See Pellestor and Paulasova, 2004. PNAs can be prepared using techniques similar to those used for automated peptide synthesis. As would a conventional nucleic acid, a PNA will hybridize with complementary DNAs or RNAs with high affinity and specificity.

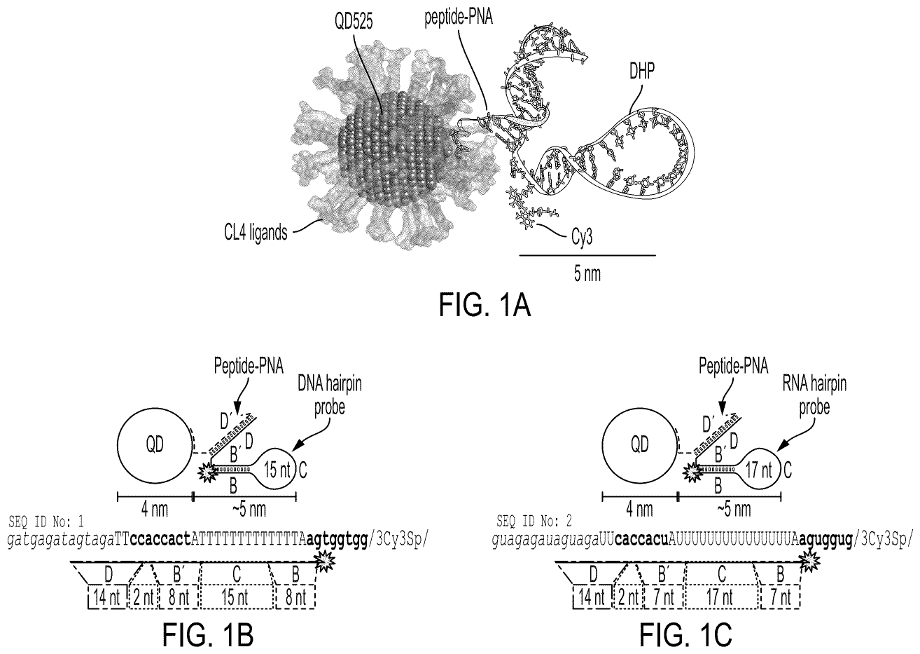

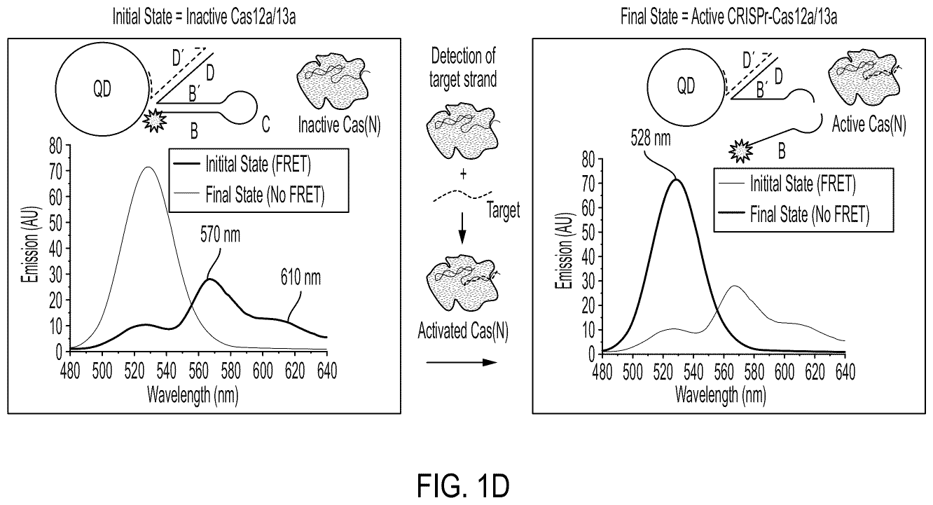

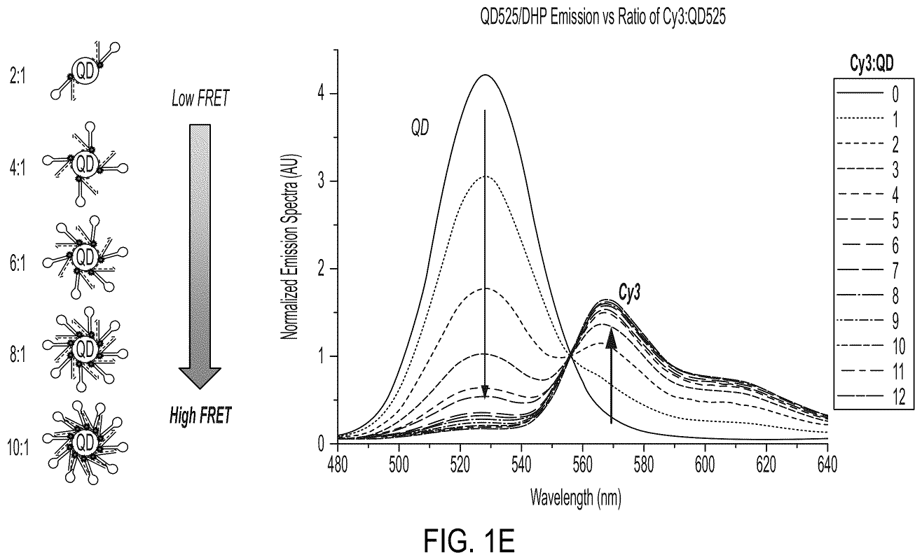

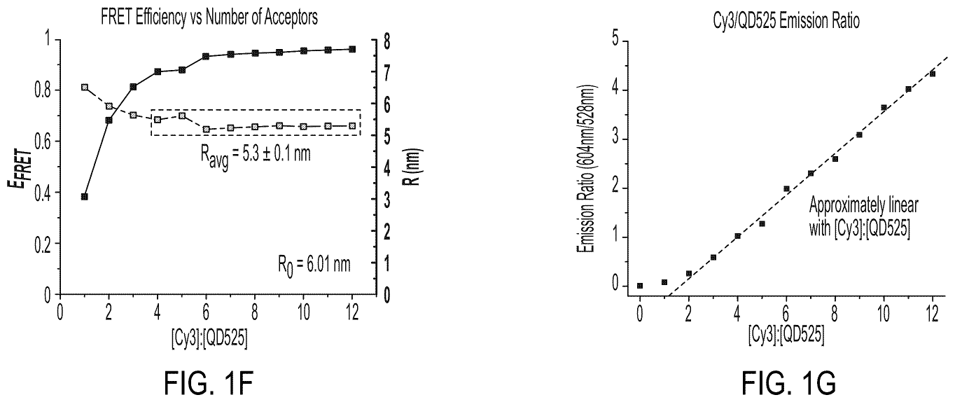

The hybrid quantum dot (QD)-nucleotide systems are designed to serve as robust ratiometric fluorescent reporters for monitoring the activity of CRISPR/Cas. As seen in FIGS. 1A-1C, the molecular beacons comprise three components: a QD, a specifically designed peptide-PNA (peptide nucleic acid), and the nucleotide hairpin.

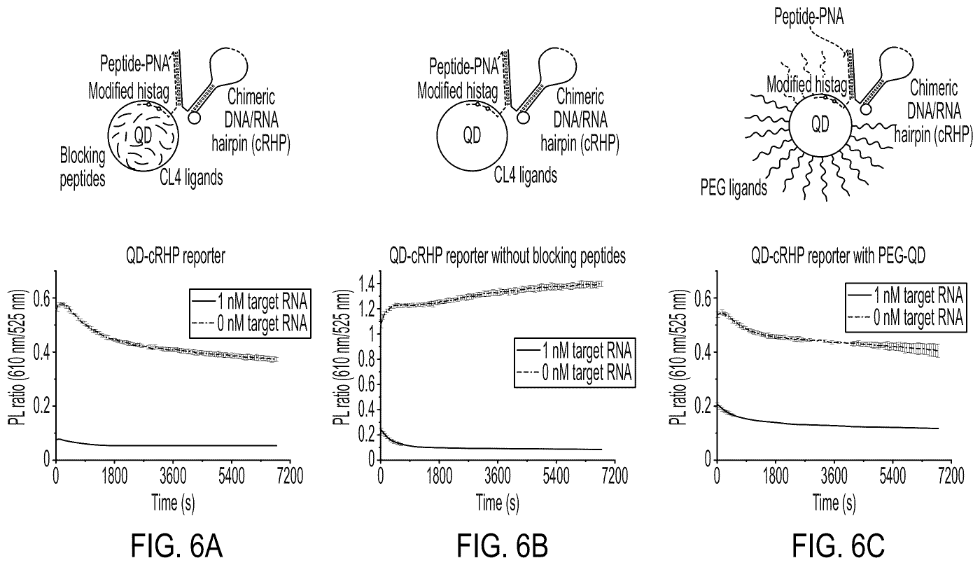

The peptide-PNA includes three functionally distinct domains: (1) the histidine tag, which is a terminal domain of the peptide comprising of a number of histidines (typically six) acting to facilitates conjugation to QDs, (2) a spacer domain that plays a role in presentation of the PNA by extending it away from the QD and is typically around six residues in length, and (3) a 14-nucleotide (nt) PNA which facilitates specific interactions with the nucleotide hairpins (depicted as D′ in FIGS. 1B and 1C). The histidine tag domain can also be extended to include additional histidines which helps to strengthen direct assembly to the QD surface of the peptide-PNA and can also function as a blocking peptide, avoiding enzyme conjugation to the QD surface (FIG. 6B).

The nucleotide hairpin can comprise DNA (and termed DHP for DNA hairpin) or RNA (and termed RHP for RNA hairpin) depending on the chosen Cas enzyme; Cas12 cleaves DNA and Cas13 cleaves RNA. The hairpin can comprise a chimeric DNA/RNA hairpin (cRHP) that can function for both Cas12 and Cas13 (FIG. 6A). The DHP and RHP are designed similarly, with a section complementary to the PNA (D in FIGS. 1B and 1C), a short spacer (2 nt in the examples depicted in FIG. 1B), then a short (in the examples, 8 or 7 nt, for DNA and RNA, respectively) section (B′) that is complementary to the final section (B) labeled with a quencher or fluorophore capable of acting as a FRET acceptor to the QD, and in between these is the exposed single stranded nucleic acid loop (C in FIG. 1) that is a substrate for and is cleaved by the activated Cas enzyme. Specifically when the target strand, which is complementary to the guide RNA (gRNA) conjugated to the Cas12 or Cas13 enzyme, is present, it activates the non-specific cleavage of single stranded oligonucleotides. Cleavage of the loop destabilizes the B′-8 binding and allows the fluorophore to diffuse from the QD surface, resulting in the ratiometric change in fluorescence.

Assembly of the sensors is based on self-assembly of the individual components and can be done in a single-pot batch approach. It was found that a particular order of addition resulted in more uniform sensors. First, the peptide-PNA is combined with the hairpin probe bridge strand (which includes a FRET quencher of the QD, such as a Cy3 dye) and allowed to anneal under suitable conditions for a period of time, such as 20 minutes. Then, the QDs are added with agitated mixing at room temperature mixing so that the polyhistidine sequence attach to the QDs. It was found that a further step of QD surface passivation with blocking peptides (such as hexahistidine) ensures optimal activity of the Cas enzymes—this can be done by incubating the QD/reporter complexes with the peptide using gentle mixing for approximately one hour at room temperature.

### Examples

Because a single QD can anchor multiple peptide-PNAs and their corresponding hairpin strands, a characterization of FRET within the QD525/DHP-Cy3 complex was made wherein the molar ratio of duplexed peptide-PNA/DHP-Cy3 (acceptor) to QD525 (donor) was varied from 0 to 12 acceptors/donor. The donor concentration was held constant at 100 nM in each solution. After assembly, the fluorescence spectra of the solutions were acquired under 350 nm excitation in a microplate reader (FIG. 1D), and the FRET efficiency was determined for each sample from donor quenching relative to an equivalent control sample. Assuming a Forster distance (Ro) of 6.0 nm, the average donor-acceptor distance (r) was determined to be 5.3±0.1 nm. Note that r values are determined from the center of the QD; considering the QD radius this signifies that the dye was found approximately 3 nm from the QD surface, well in line with the physical constraints of the system, e.g. linker lengths. The photoluminescence (PL) ratio, i.e., the FRET-sensitized fluorescence of Cy3 relative to QD525 fluorescence, was determined for each solution and plotted as a function of the molar ratio of acceptor to donor in FIG. 1E. After correcting for direct excitation of Cy3, the PL ratio was observed to be approximately linearly dependent on the number of Cy3 per QD525 for molar ratios greater than 3 Cy3/QD. Within the donor/acceptor ratios employed, the PL ratio varied by more than 100-fold between fully assembled (high molar ratio) and disassembled, and later 3D excitation and emission scans demonstrated that a 200-fold change could be achieved at as low as 5 to 1 Cy3 to QD525 with proper choice of microplate, excitation wavelength, and emission wavelengths for quantifying the QD and Cy3 emissions. Although the DHP hairpins were studied herein, the characterization results are directly translatable to the QD-RHP system as well.

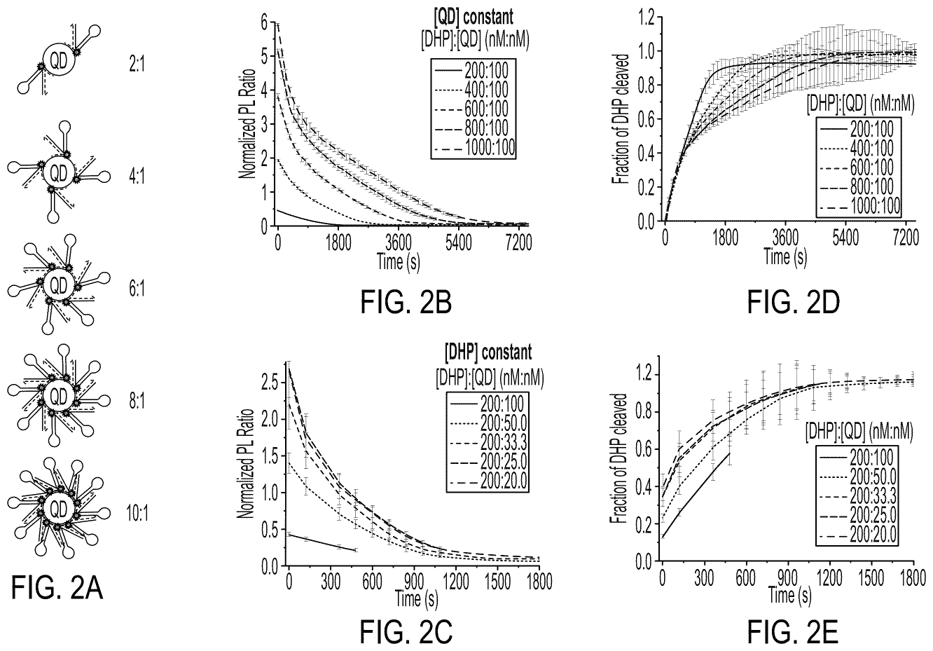

To characterize the interaction of Cas12a with DHP substrates on the QD surface, two sets of initial experiments were performed with QD-DHP reporters prepared at molar ratios of DHP-Cy3/QD525 varying from 2 to 10. First, to identify any significant effects of the QD on Cas12a activity, the QD concentration was varied in one set of experiments (FIG. 2A) and held constant in another (FIG. 2B). All reporter complexes were diluted 10× into a Cas12a reaction mixture with a final composition of 1×NEBuffer 2.1, 1 mM dithiothreitol (DTT), 50 nM Cas12a, 45 nM crRNA, 5 nm of target (template strand, TS) or non-target (non-template strand, NTS) ssDNA. QD525 concentrations ranged from 20 nM to 100 nM, and DHP-Cy3 concentrations ranged from 200 nM to 1000 nM. The reactions were maintained at 37° C. in a fluorescent plate reader, and the reaction progress was monitored every 2 min for 2 h in a 384-well plate by excitation at 380 nm and emission at 528 nm (QD donor) and 604 nm (Cy3 acceptor). Identical reaction mixtures were prepared and run in parallel with 200 nM DNaseAlert™ (DA) as a benchmark MB for comparison to QD-DHP, and reaction progress was monitored with excitation at 536 nm and emission at 556 nm.

The reactions containing the target ssDNA approached completion in less than 1 h, indicated by a plateau in the decay of the PL ratio of QD-DHP reporters and a similar plateau in the growth of the absolute intensity of DA fluorescence. Comparison to the control samples (non-target ssDNA) confirmed that Cas12a trans-cleavage was only activated in the presence of the target, and this result additionally demonstrated that the QD-DHP complexes operated successfully as reporters of the trans-cleavage activity of Cas12a/crRNA/target complexes. The initial rates of Cas12a trans-cleavage, determined from the PL ratios, were found to be independent of the QD/DHP molar ratio, suggesting that the presence of the QD neither hindered nor enhanced cleavage of the hairpin substrate. As expected, the dynamic range of each QD-DHP was found to be strongly dependent on the QD/DHP molar ratio due to the increase in the number of FRET acceptors per donor, yet the endpoints were not significantly affected by direct excitation of Cy3 for DHP-Cy3 concentrations ranging from 200 to 1000 nM.

To determine the limit of detection (LOD) of target nucleic acids with the CRISPR/Cas nucleases and QD-HP reporters, target dilution assays were performed with LbCas12a and LwCas13a. For DNA detection by LbCas12a, QD-DHP reporters were assembled at a 6 to 1 molar ratio of DHP-Cy3 to QD525 to balance between dynamic range and time to completion of the reaction. Target and non-target ssDNA dilutions were prepared and preassembled with the Cas12a/crRNA ribonucleoprotein (RNP), preheated to 37° C., and then the QD-DHP reporter or DA was added to each reaction mixture and loaded into a plate reader to monitor the reaction progress at 37° C. over 4 h. Target and non-target ssDNA dilutions varied from 5 nM to 5 pM with 100 nM of the QD-DHP reporter or 200 nM of DA. Fluorescent readout was performed every two minutes, with QD-DHPs monitored at 528 nm and 604 nm for fluorescence under 380 nm excitation, and DA monitored for 556 nm fluorescence with 536 nm excitation.

For RNA detection by LwCas13a, QD-RHP reporters were prepared at the same 6 to 1 molar ratio of acceptor to donor, RHP-Cy3 was used in place of DHP-Cy3 and RNaseAlert (RA) in place of DA. Additionally, the target RNA, a 981 nt model gene transcript (LcrV gene of Yesenia pestis), and the associated crRNA were used. Target RNA dilutions were prepared and preassembled with the Cas13a RNP at 25° C. The QD-RHP reporter or RA were loaded onto a plate reader and monitored under nearly identical conditions to the Cas12a experiments described above, and the only variation being the excitation of RA at 490 nm and emission at 520 nm. Target RNA dilutions varied from 10 nM to 1 pM with 100 nM of the QD-RHP reporter or 200 nM of RA

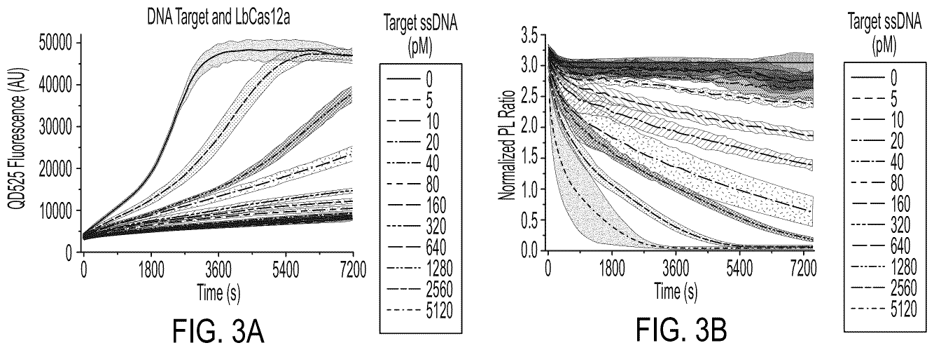

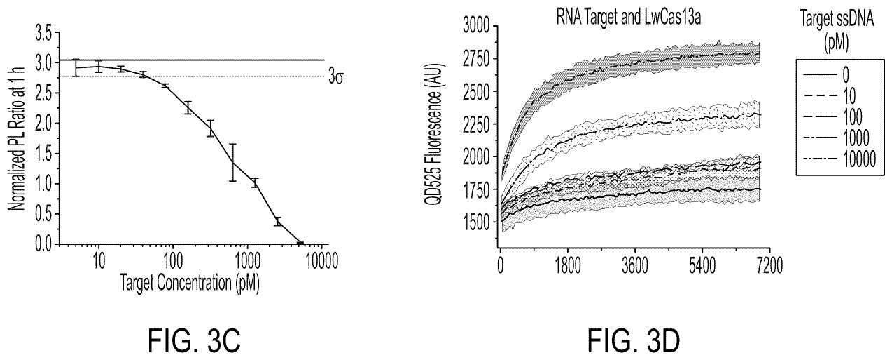

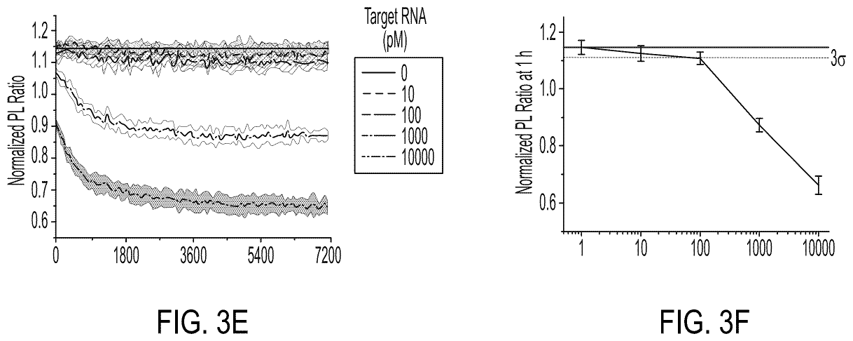

The results of each nucleic acid detection assay with QD-HP reporters were quantified using the average PL ratio of three replicates per sample normalized to the average PL ratio of 3 replicate control samples (FIGS. 3A and 3C). At target ssDNA concentrations greater than 500 pM, the QD-DHP reporters were fully cleaved by LbCas12a within the 4 h runtime of the assay. Samples with 10 nM LcrV RNA target were found to plateau within 30 minutes at approximately 20% activation of the reporters, while samples with 1 nM target approached 20% activation at 2 h. The LOD of target nucleic acids for each Cas system in combination with QD-HP reporters was determined from the normalized PL ratios after 60 min and is plotted in FIG. 3. For the detection of a short ssDNA target, the LbCas12a/QD-DHP reporter system achieved an LOD of approximately 50 pM. For the detection of the LcrV RNA target, the LwCas13a/QD-RHP reporter system achieved an LOD of 100 pM, matching the performance of RNaseAlert. It is expected that further optimization could increase the LOD further, as extended following of the assay (>24 hrs runtime) resulted in a signal discernable over noise as low as 3 pM for the LbCas12a/QD-DHP system.

To determine whether multiple nucleic acid targets could be detected with QD-HP reporters simultaneously, two spectrally distinct QD-FRET pairs were assembled for trans-cleavage by Cas12 and Cas13. A second QD-DHP reporter was assembled from a 9.4 nm diameter, commercially synthesized QD with a 625 nm emission peak (QD625 ITK-CdSe/ZnS with addition of CL4 ligands). The peptide-PNA and DHP were identical to the prior experiments but for the dye, where Cy3 was replaced with Cy5 as the FRET acceptor. The QD625:DHP-Cy5 reporter was calibrated prior to use, and the QD-DHP reporter complex was assembled at a 10 to 1 molar ratio of DHP-Cy5 to QD625 for multiplexed detection. The QD-RHP reporter was prepared using the same procedures described above for the LOD experiments at a 6 to 1 molar ratio of RHP-Cy3 to QD525. To reduce potential interference between the distinct Cas systems, LbCas12a/crRNA and LwCas13a/crRNA RNP complexes and target activated RNP complexes were assembled individually prior to combining them in the reaction mixture. For target activated solutions, the Cas, crRNA, and target nucleic acids were combined at 500 nM, 450 nM, and 100 nM in 1×NEB 2.1r buffer and 0.5 mM TCEP (tris(2-carboxyethyl)phosphine), then the LbCas12a and LwCas13a mixtures were combined and diluted 10-fold into the final reaction mixtures containing 100 nM QD525:RHP-Cy3 reporter and 20 nM QD625:DHP-Cy5 in a 384-well microplate. A 5-fold lower concentration was used in the QD625 system due to that QDs greater overall brightness under the UV excitation profile.

The microplate was maintained at 37° C. on a hot plate during preparation of the reaction mixtures, then quickly transferred to a plate reader at 37° C. to monitor the reactions. All samples were excited at 380 nm and monitored for fluorescence at 528 nm, 572 nm, 625 nm, and 670 nm, corresponding to fluorescence of QD525, Cy3, QD625, and Cy5 respectively. Four distinct samples were monitored, containing no target nucleic acids, only target RNA for Cas13, only target dsDNA for Cas12, and both target RNA and target dsDNA for activation of both Cas12 and Cas13. Four distinct control samples were prepared and monitored in parallel to the reaction mixtures, corresponding to no trans-cleavage, complete cleavage of the QD-RHP reporter, compete cleavage of the QD-DHP reporter, and complete cleavage of both QD-HP reporters. The controls were used to determine the initial and final states of each reaction and to qualitatively assess the presence of target RNA or DNA. The PL ratios of each QD-HP reporter (I_{572 nm}/I_{528 nm} and I_{670 nm}/I_{625 nm} for QD-RHP and QD-DHP, respectively) were normalized by the PL ratios of the inactive controls to identify major deviations that would signify cleavage of one or both QD-HP reporters.

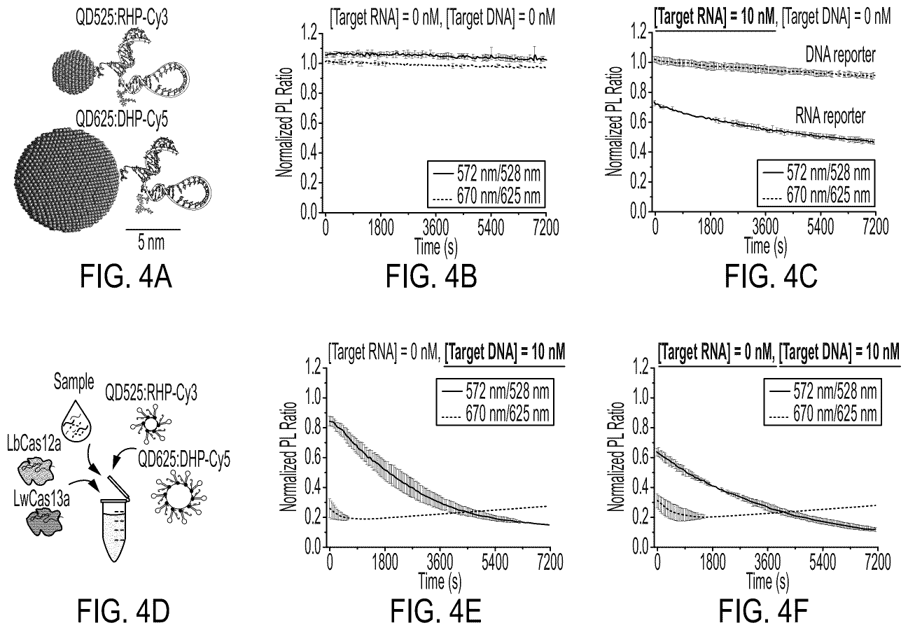

The results of the multiplexed Cas12/Cas13 assay are shown in FIGS. 4A-4F and demonstrate the ability to distinguish samples with only RNA target from samples containing only ssDNA target or no target nucleic acids. Samples including ssDNA target induced the activation of both reporter complexes, demonstrating the previously reported propensity of LbCas12a to additionally cleave RNA, albeit at a significantly reduced rate compared to ssDNA. Despite the reduced rate, target-bound Cas12 RNP cleaved more of the QD-RHP reporter than did the activated Cas13 RNP within 20 minutes due to a rapid plateau in the Cas13 activity. This behavior prevented the multiplexed system from differentiating samples containing only ssDNA targets from those containing both ssDNA and RNA targets, though this issue could be circumvented by use of another Cas12 protein that does not demonstrate trans-cleavage of RNA. Nonetheless, the results demonstrated that QD-HP reporters could be prepared for use in parallel as reporters for Cas12 and Cas13 nuclease activity, and the flexibility of the system would allow for the incorporation of other fluorescent or non-fluorescent molecules for more highly multiplexed readout.

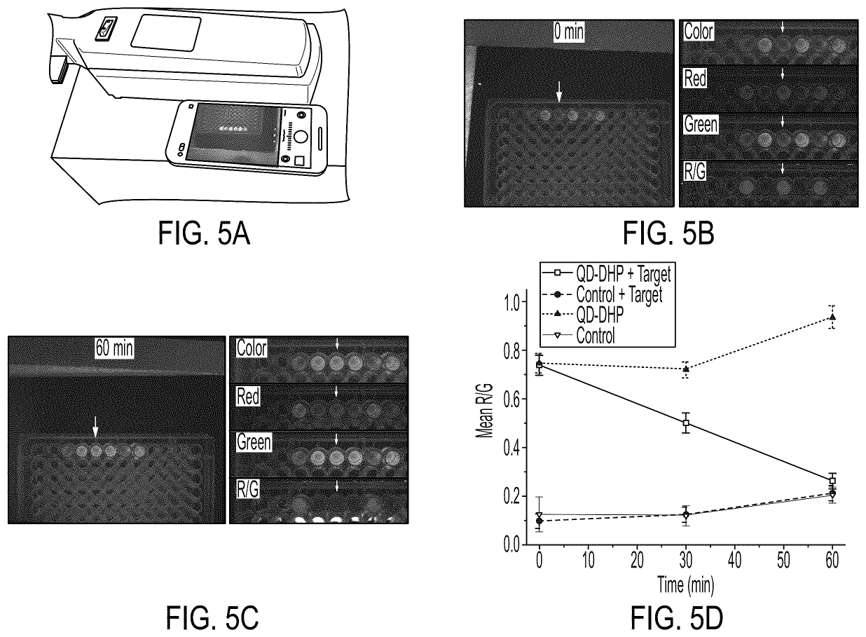

To assess the utility of QD-HP reporters for readout by a commonplace imaging device, namely a camera phone, QD525:DHP-Cy3 reporters were prepared using the protocols described previously for LOD experiments and added to a UV-transparent, 96-well microplate. Control samples for each individual component and the fully assembled and disassembled QD-DHP were also included in the plate. Cas12-RNP-target ssDNA complexes were prepared stepwise alongside a non-target control mixture, and the Cas12 mixtures were diluted 10× into the reaction mixture for final concentrations of 50 nM Cas12, 45 nM crRNA, and 5 nM target or non-target ssDNA in 1×NEB buffer. The microplate was placed in a cardboard box with windows for top-down illumination by a 5 watt, 365 nm UV lamp and for image acquisition with an IPHONE 13 Pro camera. An image was taken immediately after the addition of the Cas mixtures to the microplate, then the microplate was sealed, transferred to a hot plate, and heated to 37° C. Images were taken at 30 min and 60 min to assess the progress of the reactions, and the images are shown in FIGS. 5A through 5C.

As is clear in the unprocessed IPHONE images, the sample containing target ssDNA underwent a significant color change within the 60 min reaction timeframe, indicating successful cleavage of the QD-DHP reporters by active Cas12 nucleases. The color change was easily detectable by naked eye under UV illumination, and no optical bandpass or blue light filters were necessary for readout. To quantify the progression of QD-DHP reporter cleavage, the RGB images were separated into their component red, green, and blue channels, then pixel intensity values in the red channel (R) were divided by those in the green channel (G) to generate an R/G image roughly equivalent to the PL ratio. The R/G values reported in FIG. 5D were determined from the average and standard deviation of the values of pixels contained within the wells of each sample in the R/G microplate images for 0, 30, and 60 min. At 60 min a 300% change in the mean R/G can be observed when the target strand was present as opposed to the samples without target strand.

### Further Embodiments and Advantages

The techniques described herein can find application as an economic reporter of different CRISPR/Cas system activity in that they can be used to detect nucleotide oligomers at the nanomolar level (with a suggested attomolar detection capability). The QD-based ratiometric signal is robust and can permit simple cellphone based readout with the possibility of similar systems being constituted on paper lateral-flow assays. These types of assays are being developed for use in the field due to the unique characteristics and benefits that they can impart to sensing biological agents in resource-limited situations.

The combination of a QD donor and use of ratiometric signals is expected to provide enhanced sensitivity in and of itself, while the read-out approach is compatible with amplification and other strategies commonly used to further increase sensitivity. The QDs scaffolding capability allows for the conjugation of multiple biomolecules, which allows for multiplexing as well as added functionalities such as targeting. The brightness and stability of QDs allows for flexibility in the assay format, being feasible in both high-throughput laboratory assays as well as in resource-limited conditions, i.e. lateral flow paper assays or cell-phone imaging detection.

Simultaneous multiplexing can provide diverse information in a timely manner, reducing cost, particularly important in point-of-care (POC) applications. These applications could especially benefit from the use of a common phone camera to capture images as demonstrated herein, potentially replacing expensive custom laboratory equipment.

Use of QDs as donors in FRET-based assays bring with it a host of inherent advantages that are cumulatively unavailable to any other fluorophore in the same role, these include: (i) the ability to tune the QD emission to better match an acceptor absorption profile (i.e., an acceptor absorption maxima) while minimizing direct excitation of the acceptor (i.e., an acceptor absorption minima); (ii) QDs have high brightness and are exceptionally photostable, and with proper surface and ligand choice the QDs are very robust to other detrimental environmental conditions (e.g. temperature, solvents, pH); (iii) the technique offers the ability to array multiple acceptors around a central QD which allows tuning of the FRET efficiency by proportionally increasing the acceptor absorption cross-section, this allows for a choice of an initial FRET efficiency that provides the most dynamic changes during a subsequent sensing/reporting event; (iv) multiple QDs could be excited at a single wavelength that is distinct and significantly blue shifted from their emission—each QD can be paired with its own distinct acceptor to provide for multiplexing where the number of distinct reporters is only limited by the ability to resolve the FRET changes; (v) multiphoton excitation or the use of a further long-lifetime donor to the QD can minimize interference and background in cellular extracts; (vi) the QD surface could be used to attach multiple sensing or acceptor moieties in contrast to other organic-fluorophore based donors; and (vii) QD surface ligands can be tailored to minimize and or maximize transient interactions with enzymes, as described in Analytical and Bioanalytical Chemistry volume 407, pp. 7307-7318 (2015) and ACS Nano 2017, 11, 6, 5884-5896. The molecular beacon can operate in the presence of other surface ligands, such as PEGylated ligands, as seen in FIG. 6C.

Additional features include the following. The sensor is compatible with target strand amplification and other strategies commonly used to increase sensitivity. Due to the non-specificity of Cas12 and Cas13 activity, the sensor does not need to be modified for different target oligonucleotide. Instead, by simply changing the gRNA bound in the Cas enzyme, the system can report on the presence of different biomarkers. The same QD:peptide PNA preparation in combination with DNA and RNA hairpins can be assembled to create a Boolean logic type OR sensor against different analytes. QDs have similar broad absorbance properties but, relatively sharp emission bands, so multiplexing two sensors with a single excitation wavelength by using a QD¹:DHP and QD²:RHP can be achieved. Assembly of the QD:DHP and QD:RHP reporter can be done in a one-pot batch methodology in an aqueous buffer, avoiding a need for complex chemistry. Storage of the QD:DHP reporter can be done in mild conditions (i.e. room temperature), though extended storage would benefit from refrigeration and avoiding exposure to light. The QD:RHP will have similar shelf-life as long as the batch remains unopened, nor exposed to RNAse.

Yet further embodiments and advantages include the following. The reporter design can incorporate other nucleic acid sequences, which may be activated by other nucleases, including restriction enzymes. The design inherently lends itself to incorporation of other hydrolytic or ‘cleavage’ enzymes such as proteases or lipases by reconfiguring the recognition and or cleaved portion of the reporter to the appropriate material—this can increase the bioorthogonality and applicability of both the reporter and the sensor it is used with. The ability to access other energy transfer modalities for reporting such as electron transfer with the QD by replacing the acceptor fluorophore with an appropriate redox active electron donor or acceptor such as an electroactive ruthenium phenanthroline complex, for example. The modification of the dye-labeled hairpin to QD ratio can modify the sensor properties—controlling this ratio permits one to tailor the system to particular requirements, for example lower detection limits or greater speed of result. The ability to convert the observed change in FRET for a given reporter to quantitative units of enzyme velocity through the use of an appropriate FRET calibration curve. Due to the systems design and QD based ratiometric readout varying quantification strategies can be applied including a kinetic approach, signal end-point approach, or simple binary fluorescence color change. Potential access to increased sensing efficiency through enzyme processivity around the QD reporter rather than multiple cleavage events on independent targets which have the enzyme diffusing away between each cleavage event. The brightness and stability of QDs allows for flexibility in the assay format, making the technique feasible for both high-throughput laboratory assays as well as in resource-limited conditions using, e.g., lateral flow paper assays or cell phone imaging detection. The reporters could be lyophilized or frozen alone or with cell extracts and reconstituted or defrosted when needed. The QDs and, in turn, the reporter configuration can be excited and driven in “un-powered” modalities for field use by incorporating in BRET (bioluminescence resonance energy transfer) or CRET (chemiluminescence resonance energy transfer) driven chemical sensitization.

Regarding QDs, the examples used two forms of CdSe/ZnS QDs emitting at 525 nm and 625 nm, however QDs with other compositions and/or emission characteristics could also be used, although the presence of Zn (or other ion capable of conjugating a histag, such as Ni and Cu) in the outer layer is required for peptide-PNA binding. The QDs can be conjugated with ligands such as CL4 ligands described in commonly-owned U.S. Pat. No. 9,304,124 (see also Diaz et al, 2017).

Besides QDs, other forms of fluorescent nanoparticles could be used as long as they bind to the histidine tag. This includes nanoparticles that display Ni²⁺-nitriloacetic acid (NTA) such as described in J. Phys. Chem. C 2010, 114, 32, 13526-13531.

Also possible is the opposite configuration where a dye-labeled DNA is paired with a small quenching nanoparticle or even noble metal nanoparticle, with polyhisitidine for binding. Alternatively, thiolated PNA could link to a gold or other metal nanoparticle.

Furthermore, one could use a hybrid RNA/DNA nucleotide loop.

Still another embodiment is an extended Histag-PNA structure which eliminates the need for blocking peptides.

### CONCLUDING REMARKS

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

### Peptide Nucleic Acids

1. Pellestor, F., Paulasova, P. The peptide nucleic acids (PNAs), powerful tools for molecular genetics and cytogenetics. Eur J Hum Genet 12, 694-700 (2004).

### Quantum Dots and Ratiometric Fluorescent Readout

1. Medintz, I. L., Clapp, A. R., Mattoussi, H., Goldman, E. R., Fisher, B., Mauro, J. M. Self-assembled nanoscale biosensors based on quantum dot FRET donors. Nature Materials 2:630-638 (2003).
2. Diaz, S. A., Breger, J. C., Medintz, I. L. Monitoring Enzymatic Proteolysis Using Either Enzyme- or Substrate Bioconjugated Quantum Dots. Methods in Enzymology 571, 19-54 (2016)
3. Diaz, S. A., Malanoski, A., Susumu, K., Hofele, R. V., Oh, E., Medintz, I. L. Probing the Kinetics of Quantum Dot-Based Proteolytic Sensors. Analytical and Bioanalytical Chemistry 407, 7307-7318 (2015).
4. Hildebrandt, N., Spillmann, C. M., Algar, W. R., Pons, T., Stewart, M. H., Oh, E., Susumu, K., Diaz, S. A., Delehanty, J. B., Medintz, I. L. Energy Transfer with Semiconductor Quantum Dot Bioconjugates: A Versatile Platform for Biosensing, Energy Harvesting, and Other Developing Applications. Chemical Reviews 117, 536-711 (2017).
5. Diaz, S. A.; Gillanders, F.; Susumu, K.; Oh, E.; Medintz, I. L.; Jovin, T. M., Water-Soluble, Thermostable, Photomodulated Color-Switching Quantum Dots. Chemistry, 23 (2), 263-267 (2017).
6. Breger, J. C.; Susumu, K.; Lasarte-Aragones, G.; Diaz, S. A.; Brask, J.; Medintz, I. L., Quantum Dot Lipase Biosensor Utilizing a Custom-Synthesized Peptidyl-Ester Substrate. ACS Sensors 5, 1295-1304 (2020).

### CRISPR/Cas Biosensing

1. Gootenberg, J. S.; Abudayyeh, O. O.; Lee, J. W.; Essletzbichler, P.; Dy, A. J.; Joung, J.; Verdine, V.; Donghia, N.; Daringer, N. M.; Freije, C. A.; et al. Nucleic acid detection with CRISPR-Cas13a/C2c2. Science, 356, 438-442 (2017).
2. Qin, P.; Park, M.; Alfson, K. J.; Tamhankar, M.; Carrion, R.; Patterson, J. L.; Griffiths, A.; He, Q.; Yildiz, A.; Mathies, R.; Du, K. Rapid and Fully Microfluidic Ebola Virus Detection with CRISPRCas13a. ACS Sensors, 4, 1048-1054 (2019).
3. Hass, K. N.; Bao, M.; He, Q.; Liu, L.; He, J.; Park, M.; Qin, P.; Du, K., Integrated Micropillar Polydimethylsiloxane Accurate CRISPR Detection System for Viral DNA Sensing. ACS Omega 5, 27433-27441 (2020).
4. Broughton, J. P.; Deng, X.; Yu, G.; Fasching, C. L.; Servellita, V.; Singh, J.; Miao, X.; Streithorst, J. A.; Granados, A.; Sotomayor-Gonzalez, A.; Zorn, K.; Gopez, A.; Hsu, E.; Gu, W.; Miller, S.; Pan, C.-Y.; Guevara, H.; Wadford, D. A.; Chen, J. S.; Chiu, C. Y., CRISPR-Casl 2-Based Detection of Sars-Cov-2. Nature Biotechnology, 38, 870-874 (2020).
5. Talwar, C. S.; Park, K.-H.; Ahn, W.-C.; Kim, Y.-S.; Kwon, O. S.; Yong, D.; Kang, T.; Woo, E., Detection of Infectious Viruses Using CRISPR-Cas12-Based Assay. Biosensors, 11: 301 (2021).

### Quantum Dots as Reporters in CRISPR/Cas Assays

1. Bao, M.; Jensen, E.; Chang, Y.; Korensky, G.; Du, K., Magnetic Bead-Quantum Dot (Mb-Qdot) Clustered Regularly Interspaced Short Palindromic Repeat Assay for Simple Viral DNA Detection. ACS Applied Materials & Interfaces, 12, 43435-43443 (2020).
2. Bao, M.; Chen, Q.; Xu, Z.; Jensen, E. C.; Liu, C.; Waitkus, J. T.; Yuan, X.; He, Q.; Qin, P.; Du, K., Challenges and Opportunities for Clustered Regularly Interspaced Short Palindromic Repeats Based Molecular Biosensing. ACS Sensors 2021, 6, 2497-2522.

### Coordination of Nucleotide Hybrids to QDs

1. Bui, H.; Onodera, C.; Kidwell, C.; Tan, Y.; Graugnard, E.; Kuang, W.; Lee, J.; Knowlton, W. B.; Yurke, B.; Hughes, W. L., Programmable Periodicity of Quantum Dot Arrays with DNA Origami Nanotubes. Nano Letters, 10, 3367-3372 (2010)
2. Mathur, D.; Samanta, A.; Oh, E.; Diaz, S. A.; Susumu, K.; Ancona, M. G.; Medintz, I. L., Quantum Dot Encapsulation Using a Peptide-Modified Tetrahedral DNA Cage. Chemistry of Materials, 29, 5762-5766 (2017).
3. Green, C. M.; Hastman, D. A.; Mathur, D.; Susumu, K.; Oh, E.; Medintz, I. L.; Diaz, S. A., Direct and Efficient Conjugation of Quantum Dots to DNA Nanostructures with Peptide-PNA. ACS Nano, 15, 9101-9110 (2021).
4. Green, C. M.; Hastman, D. A.; Mathur, D.; Susumu, K.; Medintz, I. L.; Diaz, S. A. In Parameters Guiding the Self-Assembly of Quantum Dots and DNA Origami by Peptide-PNA, 2021 IEEE 21st International Conference on Nanotechnology (NANO), 28-30 Jul. 2021; pp 448-450

### QD Fluorescence in Paper Assays and Detection Using Cell Phones

1. Petryayeva, E.; Algar, W. R., Proteolytic Assays on Quantum-Dot-Modified Paper Substrates Using Simple Optical Readout Platforms. Analytical Chemistry, 85, 8817-8825 (2013).
2. Kim, H.; Tran, M. V.; Petryayeva, E.; Solodova, O.; Susumu, K.; Oh, E.; Medintz, I. L.; Algar, W. R., Affinity Immobilization of Semiconductor Quantum Dots and Metal Nanoparticles on Cellulose Paper Substrates. ACS Applied Materials & Interfaces, 12, 53462-53474 (2020).
3. Darwish, G. H.; Asselin, J.; Tran, M. V.; Gupta, R.; Kim, H.; Boudreau, D.; Algar, W. R., Fully Self-Assembled Silica Nanoparticle-Semiconductor Quantum Dot Supra-Nanoparticles and Immunoconjugates for Enhanced Cellular Imaging by Microscopy and Smartphone Camera. ACS Applied Materials & Interfaces, 12, 33530-33540 (2020).
4. Tominaga, S.; Nishi, S.; Ohtera, R., Measurement and Estimation of Spectral Sensitivity Functions for Mobile Phone Cameras. Sensors, 21, 4985 (2021).

What is claimed is:

1. A method of detecting Cas enzyme activity, the method comprising:
providing a molecular beacon comprising a quantum dot (QD), a peptide-peptide nucleic acid (PNA) bound to the QD comprising a PNA sequence and peptide comprising a polyhistidine sequence for binding to the QD, and a nucleic acid hairpin strand bound to the PNA sequence and comprising a nucleic acid having a sequence complementary to the PNA sequence, an exposed loop substrate for a Cas enzyme, and a quencher configured as a Forster resonance energy transfer (FRET) partner of the QD, wherein the combination of the peptide-PNA and the nucleic acid hairpin strand locates the quencher in a position to quench QD fluorescence;
contacting the molecular beacon with a Cas enzyme associated with a guide RNA which is in turn paired with a target RNA, thus activating the Cas enzyme; and
detecting a change in fluorescence from the QD, wherein the change results from cleavage of the exposed loop substrate by the activated Cas enzyme, resulting in dissociation of the quencher from the molecular beacon.

2. The method of claim 1, wherein the quencher is a dye.

3. The method of claim 1, performed in multiplex with at least two different exposed loop substrate sequences, each associated with a different form of QD.

4. The method of claim 1, wherein the Cas enzyme is Cas12 or Cas 13.

5. The method of claim 1, further comprising contacting the molecular beacon and the Cas enzyme with an analyte comprising the target RNA.

6. The method of claim 5, wherein the analyte is taken from an individual and the target nucleic acid is (1) a marker of a disease state or (2) from a virus or bacteria.

7. The method of claim 6, further comprising treating the individual (1) for the disease or (2) for infection by the virus or bacteria.

8. A molecular beacon comprising:
a quantum dot (QD);
a peptide-peptide nucleic acid (PNA) bound to the QD and comprising a PNA sequence and peptide comprising a polyhistidine sequence for binding to the QD; and
a nucleic acid hairpin strand bound to the PNA sequence, comprising a nucleic acid complementary to the PNA sequence, an exposed loop substrate for a Cas enzyme, and a quencher configured as a Forster resonance energy transfer (FRET) partner of the QD, wherein the combination of the peptide-PNA and the nucleic acid hairpin strand position the quencher to quench QD fluorescence,
wherein the Cas enzyme is Cas12 or Cas13.

9. The molecular beacon of claim 5, wherein the quencher is a dye.

10. A system for monitoring CRISPR (clustered regularly interspersed short palindromic repeats)/Cas activity, the system comprising:
a molecular beacon a molecular beacon comprising a quantum dot (QD), a peptide-peptide nucleic acid (PNA) bound to the QD comprising a PNA sequence and peptide comprising a polyhistidine sequence for binding to the QD, and a nucleic acid hairpin strand bound to the PNA sequence and comprising a nucleic acid complementary to the PNA sequence, an exposed loop substrate for a Cas enzyme, and a quencher configured as a Forster resonance energy transfer (FRET) partner of the QD, wherein the quencher is positioned to quench QD fluorescence; and
a Cas enzyme having a guide RNA complementary to a target nucleic acid, wherein the target nucleic acid is (1) a marker of a disease state or (2) from a virus or bacteria.
